(19) United States
(12) Patent Application Publication
(10) Pub. No.: US 2003/0032014 A1
(43) Pub. Date: Feb. 13, 2003

(54) Colony array-based cDNA library normalization by hybridizations of complex RNA probes and gene specific probes

(76) Inventors: Chia-Lin Wei, Vacaville, CA (US); Yijun Ruan, Davis, CA (US); Wenjiin Zheng, Davis, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 09/864,637

(22) Filed: May 23, 2001

(51) Int. Cl. C12Q 1/68; G01N 33/53; C07H 21/04

(52) U.S. Cl. 435/6; 536/23.2; 435/7.1

(57) ABSTRACT

Each cell normally has a widely differing number of mRNA transcribed for each gene. Consequently, a full-length cDNA library constructed from the mRNA would also have a widely differing number of cDNA for each gene. A normalized library of the full-length cDNA of a cell is useful for basic, applied, industrial, and medical research. This invention provides for a method for constructing a normalized full-length cDNA library by probing the members of a non-normalized cDNA library with a library of probes generated from mRNA in order to identify the cDNA of genes that have low or high expression. A collection of the cDNA from the library of the genes that have low expression would constitute a normalized library of these genes. This invention also provides for a method to reduce the number cDNA of genes that have high expression represented by probing these cDNA with a library of probes generated from a small randomly selected number of these cDNA. cDNA that hybridize are represented within this small randomly selected number of cDNA, while cDNA that do not hybridize are not represented. The latter cDNA can undergo further such probing to further reduce the number of cDNA represented. The cDNA from the library of the genes that have low expression and the randomly selected highly expressed cDNA would constitute a normalized library of these genes.

### FIELD OF THE INVENTION

This present invention is related to the field of molecular biology, biochemistry, genetics, and biological research, and specifically to cDNA library construction. In particular, this invention relates to a method for the construction of a normalized full-length cDNA library using a library of probes generated from the mRNA from which the cDNA library was made.

### BACKGROUND OF THE INVENTION

Large scale sequencing of cDNA libraries has been a successful and rapid approach for gene discovery. Usually thousands of clones from a cDNA library are randomly picked and sequenced for several hundreds nucleotide base pairs as expression sequence tag (EST). With this approach, it is possible to capture sequence signatures of all expressed genes of an organism. However virtually all cells have a widely differing number of mRNA transcribed for each mRNA per cell (for example, see Table 1), and hence redundant sequencing of highly abundant transcripts reduce the efficiency and increase the cost of this method for the discovery of new genes. In addition, only the EST of each gene is at hand, so that further manipulation is required in order to obtain the full length coding sequence of the gene of interest. Therefore, equalization of transcript abundance represented in a cDNA library becomes an important issue to a large scale EST sequencing project. A normalized full length cDNA library would be of greater use than a normalized EST cDNA library.

TABLE 1
Abundance classes of typical mRNA populations.

| | No. of differ- | Abundance |
|---|---|---|
| Source | ent mRNAs¹ | (molecules/cell)² |
| Mouse liver cytoplasmic | 9 | 12,000 |
| poly(A)⁺* | 700 | 300 |
| | 11,500 | 15 |
| Chick oviduct polysomal | 1 | 100,000 |
| poly(A)⁺** | 7 | 4,000 |
| | 12,500 | 5 |

*Young, et al. (1976) Biochem. 15:2823-8.
**Axel, et al. (1976) Cell 11:247-54.
¹This number refers to the number of mRNA species in a cell that have the corresponding number of mRNA molecules per cell.
²This number refers to the number molecules of each mRNA species per cell.

Efforts have been made on the normalization of cDNA libraries to particularly suit an EST sequencing project. For instance, many of the cDNA libraries used in the Washington University-Merck human EST project were normalized libraries. Current protocols for normalization of cDNA libraries were based on the re-association kinetics of nucleic acids. Although some successes have been reported, these procedures are complicated, tedious and technical demanding, resulting many non-successful experiences. In addition to these technical difficulties, these methods have some serious problems during manipulations, such as size bias toward short clones and reduction of clone representations after rounds of library amplifications. This bias towards short clones are a major defect for full-length cDNA cloning in those normalized libraries. As of today, large scale cDNA sequencing programs have only a 10% efficiency to isolate unique transcripts, and full-length transcripts of many genes, particularly the rare genes, have not been captured in human and other model organisms. The importance of full-length cDNA libraries are widely recognized and some works are currently ongoing (Rubin, G. M., Hong, L., Brokstein, P., Evans-Holm, M., Frise, E., Stapleton, M., and Harvey, D. A., “A Drosophila Complementary DNA Resource”, Science 287:2222-4, 2000; The RIKEN Genome Exploration Research Group Phase II Team and the FANTOM Consortium, “Functional annotation of a full-length mouse cDNA collection”, Nature 409:685-90, 2001).

Mangiarotti, et al. (Mangiarotti, G., Chung, S., Zuker, C., and Lodish, H. F., “Selection and analysis of cloned developmentally-regulated Dictyostelium discoideum genes by hybridization-competition”, Nucleic Acids Res. 9:947-63, 1981) disclose a technique for selection of cloned gene segments which are expressed preferentially at one developmental stage but at a relatively low level. Mangiarotti, et al. disclose probing cloned genomic DNA but do not teach or suggest probing a cDNA library. Mangiarotti, et al. do not disclose constructing a normalized cDNA library.

Sasaki, et al. (Sasaki, Y. F., Iwasaki, T., Kobayashi, H., Tsuji, S., Ayusawa, D., and Oishi, M., “Construction of an equalized cDNA library from human brain by semi-solid self-hybridization system”, DNA Res. 1:91-6, 1996) and Tanaka, et al. (Tanaka, T., Ogiwara, A., Uchiyama, I., Takagi, T., Yazaki, Y., and Nakamura, Y., “Construction of a normalized directionally cloned cDNA library from adult heart and analysis of 3040 clones by partial sequencing”, Genomics 35:231-5, 1996) disclose a method of eualizing an cDNA library by self-hybridizing cDNA with poly(A)⁺ RNA (with the cDNA in a large excess) and removing the RNA-DNA complexes. This method relies on the RNA-DNA hybridization taking place with all the species and members of the cDNA unseparated.

Soares, et al. (Soares, M. B., Bonaldo, M. D. F., Jelene, P., Su, L., Lawton, L., and Efstratiadis, “Construction and characterization of a normalized cDNA library”, Proc. Natl. Acad. Sci. USA 91:9228-32, 1994), Bonaldo, et al. (Bonaldo, M. D. F., Lennon, G., and Soares, M. B., “Normalization and subtraction: two approaches to facilitate gene discovery”, Genome Res. 6:791-806, 1996), Bonaldo, et al. (U.S. Pat. No. 5,702,898; 1997) and Soares, et al. (U.S. Pat. No. 5,846,721; 1998) disclose methods to normalize a cDNA library by converting the cDNA library into ss circles, generating complementary polynucleotides to the ss circles, hybridizing the ss circles to the complementary polynucleotides to produce partial duplexes, and separating the unhybridized ss circles from the hybridized ss circles. None of these references disclose hybridizing a probe library constructed using mRNA templates to a cDNA library in order to identify cDNA clones that are expressed at low amounts, and pooling or collecting them to form a normalized cDNA library.

Schena, et al. (Schena, M., Shalon, D., Heller, R., Chai, A., Brown, P. O., and Davis, R. W., “Parallel human genome analysis: microarray-based expression monitoring of 1000 genes”, Proc. Natl. Acad. Sci. USA 93:10614-9, 1996) disclose a microarray containing 1,046 human cDNAs of unknown sequences blotted with human mRNA labeled with fluorescein and Cy5-dCTP in order to identify known and novel heat shock and phorbol ester-regulated genes in human T-cells. Schena, et al. do not disclose identifying cDNA clones that are expressed at low amounts and pooling or collecting them to form a normalized cDNA library.

Carninci, et al. (Carninci, P., Shibata, Y., Hayatsu, N., Sugahara, Y., Shibata, K., Itoh, M., Konno, H., Okazaki, Y., Muramatsu, M., and Hayashizaki, Y., “Normalization and subtraction of cap-trapper-selected cDNAs to prepare full-length cDNA libraries for rapid discovery of new genes”, Genome Res. 10:1617-30, 2000) describe a method of preparing normalized and subtracted cDNA libraries by hybridizing the first-strand, full-length cDNA with several RNA drivers, including starting mRNA as the normalizing driver and run-off transcripts from mini-libraries containing highly expressed genes, rearrayed clones, and previously sequenced cDNAs as subtracting drivers. Carninci, et al. disclose using biotinylated RNA from cellular mRNA and already collected cDNA to hybridize and remove abundant and already collected cDNA. The method of Carninci, et al. relies on this RNA-DNA hybridization taking place with all the species and members of the cDNA unseparated.

Wiemann, et al. (Wiemann, et al., “Toward a catalog of human genes and proteins: sequencing and analysis of 500 novel complete protein coding human cDNAs”, Genome Res. 11:422-35, 2001) disclose a library of 500 novel complete human cDNA clones. Wiemann, et al. do not disclose a method of constructing a normalized cDNA library by hybridizing a probe library constructed using mRNA templates to a cDNA library.

The invention disclosed here will make it possible to collect all rare genes in cDNA clones in a very efficient and effective way. With this invention, we array full-length cDNA library colonies onto nylon filters in high-density, and hybridize the filter with complex RNA probes derived from the same set of RNA that was used for the library construction. From 100,000 arrayed clones, over 30,000 to 40,000 low abundant clones can be selected from one hybridization experiment. Since the low abundant clones are all toward the low end of redundancy, the frequency of representation of each of these clones is close to equal in the arrayed library. If the starting cDNA libraries contain a high percentage of full-length cDNA clones, then about 80-90% of the total clones would be full-length (i.e., about 80-90% of the 100,000 total clones are full-length). Consequently, over 20,000 unique full-length genes can be captured from any organisms in one experiment (since about 80-90% of over 30,000 to 40,000 low abundant clones is more than 20,000 clones). This invention will, therefore, greatly reduce the cost in sequencing of large number of highly redundant cDNA clones and obtain full-length functional clones with minimal redundancy.

### SUMMARY OF THE INVENTION

The present invention provides for a method for constructing a normalized cDNA library of genes of low expression, comprising: (a) constructing a non-normalized cDNA library from an RNA sample, wherein said RNA sample contains different species of RNA of different amounts, wherein each member of said non-normalized cDNA library is separate from other members; (b) identifying the relative amounts of each member of said non-normalized cDNA library represented in said RNA sample; (c) pooling the members of said group of members of said non-normalized cDNA library represented in low amounts by said RNA sample in a collection; whereby said collection is said normalized cDNA library of genes of low expression.

The invention also provides for a method for constructing a normalized cDNA library, comprising: (a) constructing a non-normalized cDNA library from an RNA sample, wherein said RNA sample contains different species of RNA of different amounts, wherein each member of said non-normalized cDNA library is separate from other members; (b) identifying the relative amounts of each member of said non-normalized cDNA library represented in said RNA sample; (c) dividing the members of said non-normalized cDNA library into groups; wherein one group of members of said non-normalized cDNA library is represented in low amounts by said RNA sample and one or more groups of members of said non-normalized cDNA library is represented in high amounts by said RNA sample; (d) selecting one group of said one or more groups of members of said non-normalized cDNA library represented in high amounts by said RNA sample; (e) identifying the members in said group of members that is not represented within a sub-group of members selected from said group of members; (f) forming a group of members from the members identified in step (e) and repeating step (e) until every member of said group of members has been selected within a sub-group of members;(g) repeating steps (d)-(f) with every group of said one or more groups of members of said non-normalized cDNA library represented in high amounts by said RNA sample; (h) pooling the members of said group of members of said non-normalized cDNA library represented in low amounts by said RNA sample and the members of every sub-group selected in a collection; whereby said collection is said normalized cDNA library.

Another aspect of the invention is a method of identifying the relative amounts of each member of a non-normalized cDNA library represented in an RNA sample comprising: separating the members of said non-normalized cDNA library, constructing a labeled probe library from said RNA sample; hybridizing the labeled probe library to said non-normalized cDNA library, whereby there is a differential of the amount of labeled probe of said labeled probe library hybridized to each individual member of said non-normalized cDNA library; and, identifying the individual members of said non-normalized cDNA library hybridized with low amounts of labeled probe.

Another aspect of the present invention is any normalized cDNA library constructed using any of the methods of the present invention. Preferably, the normalized cDNA library is a normalized full-length cDNA library.

### BRIEF DESCRIPTION OF THE FIGURES

FIG. 1 depicts filter hybridization by complex RNA probes. The alkali lysed and fixed colony filter was hybridized by the labeled probe library comprising the complex probes of first strand cDNA derived from the RNA sample. After the hybridized filter was exposed to the phosphor screen for a few days, the screen was scanned, and the data was captured into the computer. The subsequent image and data were then analyzed using ArrayVision™. The circles define the spots of clones imprinted or center spots devoid of colony. A primary 3×3 imprinting unit was used so that the center spot of each unit is devoid of colony and its hybridization intensity or signal values were used for local background noise subtraction. The colony spots have higher hybridization signals represented abundant clones, the colony spots with medium signals represented clones in medium abundant class, while the colony spots with low signals represented rare clones in the low abundance class.

FIG. 2 depicts the abundance distribution of clones revealed by complex RNA probes. After hybridization data was analyzed using ArrayVision™, all clones were sorted based on hybridization signal intensities and plotted accordingly. The relative signal intensity reflecting the abundance of each clone is plotted on the ordinate (y-axis). The clones in the order of corresponding intensities were plotted on the abscissa (x-axis). About 1,000 clones have very low hybridization signals, and therefore considered as rare clones in this library.

FIG. 3 depicts a flowchart of two embodiments the cDNA normalization method. The thick arrows represent the sequential steps in one embodiment of a method for constructing a normalized cDNA library of genes of low expression. The thin arrows represent the additional steps, in addition to the steps of the method for constructing a normalized cDNA library of genes of low expression, of one embodiment of another method for constructing a normalized cDNA library. Both embodiments of the methods start with an RNA from a biological sample, from which a cDNA library is constructed (preferably full-length, and preferably in a plasmid cloning vector). Each colony arising from each member of the cDNA library is picked so that the colonies are arrayed on one or more plates. All colonies are glycerol archived. From each plate is produced a high density colony filter, which is subjected to alkali filter treatment to fix the DNA from each colony onto the filter. In parallel, a set of complex probes constructed from the RNA sample is constructed (preferably the probes are first strand cDNA labeled with ³²P). The complex probes are hybridized to the DNA fixed on the filter. The low abundance clones are identified, based on their low hybridization signals, and selected. This collection of selected clones represents a normalized cDNA library of genes of low expression. An additional alternate step is the sequencing of all the clones in the collection to determine and/or ensure there are no redundant clones. In addition, from the results of the hybridization of the complex probes to the DNA fixed on the filter, the high and medium abundance clones can be identified, based on their low hybridization signals, and selected. From each group of the high or medium abundance clones, a sub-population of clones, such as 1,000 out of 33,000 clones, can be chosen and sequenced to identify non-redundant clones. The non-redundant clones identified from the sub-population of clones can be used to construct a set of mixed clone specific probes. The mixed clone specific probes are used to hybridize to the clones not chosen from the group of clones from which the sub-population of clones was chosen. The clones that do not hybridize to the mixed clone specific probes are identified. The low abundance clones previously selected, the sub-population of clones chosen, and the clones that do not hybridize to the mixed clone specific probes put together in a collection represent a normalized cDNA library of genes. An additional alternate step is the sequencing of all the clones in the collection to determine and/or ensure there are no redundant clones.

### DESCRIPTION OF THE SPECIFIC EMBODIMENTS

Definitions

The word “about”, when applied to a number, is defined to encompass any number closer to the number (which the word “about” applies to) to the last significant digit of that number than to another number with a different integer at the same last significant digit (e.g. “about 10” equals x, where 9.5≦x<10.5).

The Invention

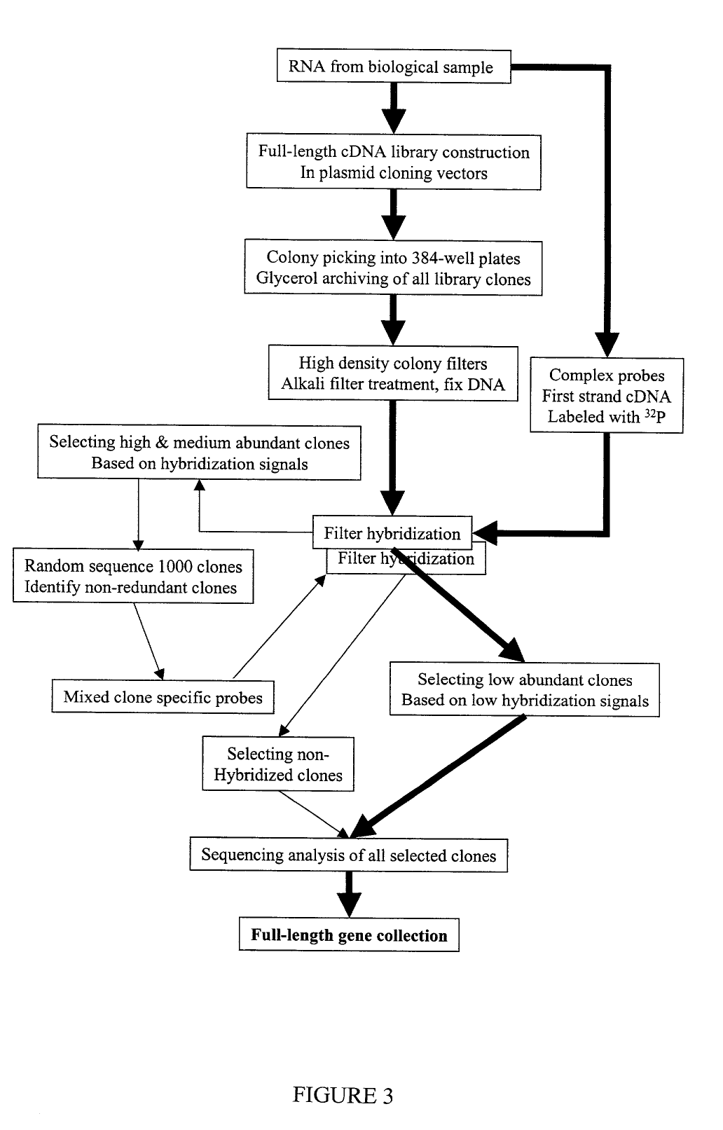

The present invention provides for a method for constructing a normalized cDNA library of genes of low expression, comprising: (a) constructing a non-normalized cDNA library from an RNA sample, wherein said RNA sample contains different species of RNA of different amounts, wherein each member of said non-normalized cDNA library is separate from other members; (b) identifying the relative amounts of each member of said non-normalized cDNA library represented in said RNA sample; (c) pooling the members of said group of members of said non-normalized cDNA library represented in low amounts by said RNA sample in a collection; whereby said collection is said normalized cDNA library of genes of low expression. One embodiment of this method is exemplified by the steps in thick arrows in the flowchart of FIG. 3.

The invention also provides for a method for constructing a normalized cDNA library, comprising: (a) constructing a non-normalized cDNA library from an RNA sample, wherein said RNA sample contains different species of RNA of different amounts, wherein each member of said non-normalized cDNA library is separate from other members; (b) identifying the relative amounts of each member of said non-normalized cDNA library represented in said RNA sample; (c) dividing the members of said non-normalized cDNA library into groups; wherein one group of members of said non-normalized cDNA library is represented in low amounts by said RNA sample and one or more groups of members of said non-normalized cDNA library is represented in high amounts by said RNA sample; (d) selecting one group of said one or more groups of members of said non-normalized cDNA library represented in high amounts by said RNA sample; (e) identifying the members in said group of members that is not represented within a sub-group of members selected from said group of members; (f) forming a group of members from the members identified in step (e) and repeating step (e) until every member of said group of members has been selected within a sub-group of members;(g) repeating steps (d)-(f) with every group of said one or more groups of members of said non-normalized cDNA library represented in high amounts by said RNA sample; (h) pooling the members of said group of members of said non-normalized cDNA library represented in low amounts by said RNA sample and the members of every sub-group selected in a collection; whereby said collection is said normalized cDNA library. One embodiment of this method is exemplified by the steps in thick and thin arrows in the flowchart of FIG. 3.

Another aspect of the invention is a method of identifying the relative amounts of each member of a non-normalized cDNA library represented in an RNA sample comprising: separating the members of said non-normalized cDNA library, constructing a labeled probe library from said RNA sample; hybridizing the labeled probe library to said non-normalized cDNA library, whereby there is a differential of the amount of labeled probe of said labeled probe library hybridized to each individual member of said non-normalized cDNA library; and, identifying the individual members of said non-normalized cDNA library hybridized with low amounts of labeled probe.

Another aspect of the present invention is any normalized cDNA library constructed using any of the methods of the present invention. Preferably, the normalized cDNA library is a normalized full-length cDNA library.

The RNA sample can be obtained from any source containing RNA. The source can be biological. The source can be cellular. Examples of cellular sources being a cell, a group of cells, a tissue, a cell culture, an organ, a whole organism, or any part of an organism that contains mRNA. The RNA sample can also be obtained from different cellular sources, or different cell types or tissues of the same organism, or from cells of different organisms. The RNA sample can be a mRNA sample. The RNA sample can be a whole mRNA preparation from a source, or mRNA of a specific criteria from a source, for example, only mRNA of a specific size or specific nucleotide sequence. mRNA of a specific size or range of sizes can be obtained by passage of the RNA sample through a size-fractionating column or gel or any other means known in the art. The RNA sample comprises mRNA, messages, transcripts, or transcriptional products from a source. Each mRNA molecule is transcribed from a gene. Each gene has a promoter sequence, a coding portion, and a terminator sequence. The promoter sequence of each gene directs the transcription of the coding portion of that gene. The promoter sequence can also contain sequences important for the regulation of the transcription of the gene. Eukarytoic genes can contain one or more introns and one or more exons. After the mRNA of an eukaryotic gene is transcribed, the mRNA can undergo one or more splicing events to delete the intron(s) and connect the exons. Eukaryotic genes that have only one exon do not have any intron and do not undergo any splicing. The spliced mRNA is termed a processed or mature mRNA. The mature mRNA has the sense codons directly linked without any intervening introns. The eukaryotic mRNA has a poly(A)⁺ tail. The poly(A) ⁺ tail is a common nucleotide sequence found at the 3′ end of all eukaryotic mRNA.

There are at least four different variables affecting the relative abundance of a species of mRNA in an RNA sample: the species of the organism from which the sample is taken, the genotype of specific the organism from which the sample is taken, the cell type from which the sample is taken, and the time or stage of development from which the sample is taken. The genes of different species of organisms are different from each other. In addition, within the same species of organism there is a variation in genotype which can affect the types of genes and the transcription of each gene. Multi-cellular organisms have different cell types within each organism. Within each species of organism, each different cell type transcribes a different set of genes. Temporally, at different stages of development of each organism or each cell, each cell transcribes a different set of genes. Different genes within an RNA sample would have different relative amounts or abundances. Therefore, each gene or clone of each gene (or clone) would have different relative amounts or abundances (see Table 1).

Typically the source of the RNA sample is derived from a source containing DNA from which RNA is transcribed. Preferably the DNA is genomic DNA. The transcription of the genomic DNA can take place in vitro or in vivo. The source or genomic DNA is derived or obtained from a cellular or non-cellular organism. A non-cellular organism can be a virus. Preferably the source is cellular. Cellular sources are eubacteria, archaebacteria, or eukaryotic cells or organisms. Preferably, the cellular source is eukaryotic, because all eukarytoic transcripts have a common nucleotide sequence at the 3′ end of every transcript: a poly(A)⁺ tail. The eukaryotic source can be a plant or animal. The plant is any plant, especially commercially valuable plants such as soy, tobacco, wheat, rice, or corn. The animal is any animal, such human, ape, mouse, rat, cow, pig, horse, goat, sheep, dog, cat, chicken, zebrafish, or fruitfly. The human cell can be any human cell, such as a human kidney cell.

General molecular biology procedures, such DNA or RNA extraction, DNA or RNA purification, DNA or RNA size fractionation, hybridization, DNA sequencing, etc., are known in the art (Sambrook, et al., Molecular Cloning: A Laboratory Manual (2d ed.), Vols. 1-3, Cold Spring Harbor Laboratory Press, Plainview, N.Y., 1989). Kits for total RNA isolation from a cell are available commercially (for example, S.N.A.P.™ Total RNA Isolation Kit, Invitrogen, Carlsbad, Calif.). Poly(A)⁺ RNA can be isolated from total RNA using kits available commercially (for example, mRNA Separator Kit, Clontech Laboratories, Inc., Palo Alto, Calif.).

Within each library, whether non-normalized or normalized, each library comprises individual molecules or “members” or “clones”, and each library comprises molecules of specific nucleotide sequences (notwithstanding the number of adenine in the poly(A)⁺ tail) or “species”. Within each library, there can be “species” comprising of only one “member”, and “species” comprising of many “members”. Each species typically represents the product (transcriptional or otherwise) of one gene or structural gene or open reading frame (“ORF”).

A non-normalized cDNA library can be constructed or synthesized from an RNA sample. The RNA sample preferably comprises a mRNA preparation from a cell. Preferably, a commercially available total RNA preparation is used. The mRNAs are converted into double-stranded (ds) cDNA in vitro using reverse transcriptase to synthesize complementary cDNA strands from the mRNA template. In order to obtain ds DNA suitable for ligation into a vector, the ds cDNA copy of the mRNA can be methylated and equipped with suitable (such as EcoRI) linkers. Methods for methylation of DNA are well known in the art, and involve the use of commercially available methylases that covalently join methyl groups to adenine or cytosine residues within specific target sequences. In the process of converting mRNA into ds cDNA in vitro, a first strand is synthesized by the reverse transcriptase and separated from the mRNA by treatment with alkali or using a nuclease such as RNaseH. This step can be achieved using a reverse transcriptase that also has RNaseH activity. Escherichia coli DNA polymerase then uses the first cDNA strand as template for the synthesis of the second cDNA strand, thereby producing a population of ds cDNA molecules from the original poly(A)⁺ mRNA.

The non-normalized cDNA library can be constructed or synthesized with the cDNA insert in a vector. A vector can comprise a cloning vector. A vector can comprise a plasmid. Each member of a non-normalized library comprises a cDNA insert in a vector, such that the members can different cDNA inserts each inserted in a vector, wherein the same vector is used throughout the entire library. The non-normalized cDNA library can be a non-normalized full-length cDNA library. The relative abundance of each species of cDNA is proportional to the relative abundance of each RNA species within the RNA sample. The vector can be amplified by an eukaryotic host cell, by a prokarytoic host cell, or by both. The suitability of a vector depends on the nucleotide sequences found within the vector. A suitable prokaryotic host cell is a bacteria, such as E. coli. For example, a vector with an origin of DNA replication and a selectable marker (such as an antibiotic resistance marker, such as the ampicillin resistance gene from Tn3) can be amplified using E. coli. A suitable eukaryotic host cell is a yeast, such as Saccharomyces cerevisiae. For example, a vector with the 2μ circle plasmid sequence and a selectable marker (such as the URA3 gene) can be amplified using S. cerevisiae. Depending on the desired host to be used, the necessary nucleotide structures necessary for maintenance in the host, such as origin of replication sites, amplifiable selectable markers, etc., and expression in the host, such as promoters, activation sites, etc. need to be present on the vector. Such construction or synthesis are well known to one of ordinary skill of the art (see Old and Primrose, Principles of Gene Manipulation 5th ed., Blackwell Science, Oxford, U.K., 1994; Sambrook, et al., Molecular Cloning: A Laboratory Manual (2d ed.), Vols. 1-3, Cold Spring Harbor Laboratory Press, Plainview, N.Y., 1989).

The relative number of cDNA members of each species in a cDNA library constructed is proportional to the relative number of RNA members of each species in a RNA sample from which the cDNA library is constructed. The relative number of cDNA members of each species in the non-normalized cDNA library constructed is proportional to the relative number of RNA members of each species in the RNA sample from which the non-normalized cDNA library is constructed.

The method can further comprise introducing each member of said non-normalized cDNA library into a host cell, wherein said introducing step is subsequent to said constructing and prior to said hybridizing. The method can also further comprise amplifying each member of said non-normalized cDNA library, wherein said amplifying comprises growing each said host cell containing a member, wherein said amplifying step is subsequent to said introducing and prior to said hybridizing. The host cells can be grown on or in any liquid or solid media. Preferably, the host cells are grown on a solid media. The host cells can be grown on membranes, on plates, in array on plates, or on any other solid support. When grown in array, the arrays can be in high density. Preferably, the host cells are grown in high-density array. When host cells are grown on a solid surface, a pure colony or colony spot is produced. Each colony or colony spot encompasses clones of the same member. The introduction of each member into a suitable host cell is preferably a transformation wherein the host cell is prior to transformation made competent for transformation. Methods of transformation and making cells competent are well known to one of ordinary skill in the art.

Each member of the cDNA library can be separated from each other member. The separation can take place (1) by separating the members of the cDNA library and introducing each member into a host cell, or (2) by introducing the members of the cDNA library, mixed together, into a culture or group of the host cells and then separating each cell containing a member on a solid media suitable and permissive for growth of a host cell containing a member (but not permissive for growth of a host supplemented with an antibiotic to prevent growth of host cells not containing a member, or the cell not containing a member). Growth of a member of the cDNA library in a host cell in a media, either liquid or solid, results in amplification of the member of the cDNA library, which means the amplification of the cDNA insert of each member. The media can be media that lacks an essential nutrient to prevent growth of host cells not containing a vector that permits growth on such a media.

The cDNA insert can be flanked on both ends by restriction sites that when digested have sticky ends. These restriction sites can be unique such that there is one unique restriction site on one end of the cDNA insert and another unique restriction site on the other end; in order to facilitate directional cloning. Alternatively, both ends can have the same restriction site. Alternatively, the cDNA insert prior to insertion into the vector can have blunt ends suitable for blunt end ligation into a vector that has blunt ends. Alternatively, there can be a sticky end at one end and a blunt end at the other; in order to facilitate directional cloning.

Premade pure, intact, total RNA from human, mouse, or rat are available commercially (Ambion, Austin, Tex.). Amplified murine cDNA libraries from mouse testis, lung, pancreas, mammary tumor, skeletal muscle, liver, brain, heart, kidney, fetal brain, and spleen; and from rat brain, spleen and fetal brain are available commercially (Edge Biosystems, Gaithersburg, Md.). Amplified human cDNA libraries from the lung, bone marrow, fetal kidney, pancreas, placenta, umbilical vein endothelial, pituary, fetal liver, mammary, lymphoma (Raji cells), trachea, thymus, adrenal, skeletal muscle, uterus, small intestine, lymph node, prostate, T-cell (activated), liver, thyroid, fetal brain, stomach, brain, heart, fetal lung, spinal cord, stimulated T-cell leukemia (THF-stimulated Jurkat cells), kidney, and spleen are available commercially (Edge Biosystems, Gaithersburg, Md.). Unamplified human and murine cDNA libraries are also available commercially (Edge Biosystems, Gaithersburg, Md.).

The constructing step can comprise catalyzing a reverse transcription reaction for each species of said RNA sample, wherein said catalyzing takes place under conditions permissible for catalyzing a reverse transcription reaction. The catalyzing step can comprise: (i) hybridizing poly-T oligonucleotide primers to said RNA sample; (ii) adding dATP, dCTP, dGTP, dTTP, and reverse transcriptase; and (iii) incubating said RNA sample at a temperature permissible for catalyzing a reverse transcription reaction. Alternatively, if a normalized cDNA library of a set of genes that contain a length of nucleotides that is identical among these genes but not found in other genes is desired, then the poly-T oligonucleotide primers can be replaced with a set of oligonucleotide primers with a nucleotide sequence complementary to the length of nucleotides that is identical among these genes. Certain nucleotide residue position(s) within the nucleotide sequence complementary to the length of nucleotides that is identical among these genes may be made degenerate.

The identifying of step (b) can comprise: (i) constructing a labeled probe library from said RNA sample; (ii) hybridizing said labeled probe library to said non-normalized cDNA library; (iii) identifying the relative amounts of labeled probe hybridized to each member of said non-normalized cDNA library. The labeled probe library is a complex probe library in that the different species of probes are of unequal amount. The amount of each species of probe is proportional to the amount or abundance of that species of RNA in the RNA sample. This labeled probe library is also termed “complex RNA probes” in that it is “complex” because different species of probes are of unequal amount, and it is “RNA” because the probes are derived from RNA sequences.

The constructing of the labeled probe library can comprise subjecting the RNA sample to a reverse transcription reaction using a poly-T primer, dNTP, and reverse transcriptase. The probe library can be labeled by either using labeled poly-T primer or labeled dATP, dCTP, dGTP, and/or dTTP. The type of label can comprise poly-T primer, dATP, dCTP, dGTP, and/or dTTP with one or more radioactive isotope, fluorescence, chemiluminescent label, or the like. Preferably, the constructing is one that does not favor the synthesis of a probe from one mRNA species, with the common nucleotide sequence, over the synthesis of a probe from another mRNA species, with the common nucleotide sequence.

The cDNA members can be immobilized, fixed, attached or bound to any solid support, such as a filter membrane, nitrocellulose membrane, a nylon membrane, DBM-cellulose, APT-cellulose, or any other suitable solid support. The binding can comprise hydrophobic interactions or covalent bonds. Methods of such immobilizing, fixing, attaching or binding are well known in the art. Methods of hybridization of any probes to any nucleic acid are well known in the art. Preferably, hybridization is performed under a stringent condition, since only polynucleotides with complementary sequences are sought to be hybridized to each other. One of ordinary skill of the art can determine the conditions and level of stringency required to perform the hybridization. Also, preferably, hybridization is in situ hybridization. (See Sambrook, et al., Molecular Cloning: A Laboratory Manual (2d ed.), Vols. 1-3, Cold Spring Harbor Laboratory Press, Plainview, N.Y., 1989.)

The hybridization conditions between probe and cDNA member should be selected such that the specific recognition interaction, i.e., hybridization, of the two groups of molecules is both sufficiently specific and sufficiently stable (see, for example, Hames and Higgins, Nucleic Acid Hybridisation: A Practical Approach, IRL Press, Oxford, 1985). These conditions are dependent on both the specific sequences and the guanine and cytosine (GC) content of the complementary hybrid strands. The conditions may often be selected to be universally equally stable independent of the specific sequences involved. This typically will make use of a reagent such as an alkylammonium buffer (see, Wood, et al., “Base composition-independent hybridization in tetramethylammonium chloride: a method for oligonucleotide screening of highly complex gene libraries,” Proc. Natl. Acad. Sci. USA, 82:1585-8, 1985; and Krupov, et al., “An oligonucleotide hybridization approach to DNA sequencing,” FEBS Lett., 256:118-22, 1989; each of which is hereby incorporated herein by reference.) An alkylammonium buffer tends to minimize differences in hybridization rate and stability due to GC content. Temperature and salt conditions along with other buffer parameters should be selected such that the kinetics of renaturation should be essentially independent of the sequence involved. In order to ensure this, the hybridization reactions should be performed in a single incubation of all the substrate matrices together exposed to the identical same target probe solution under the same condition. Control hybridizations should be included to determine the stringency and kinetics of hybridization.

Any suitable form of labeling of the probes can be used. A quickly and easily detectable signal is preferred. Suitable labels are fluorescent labels, heavy metal labels, chemiluminescent labels, magnetic probes, chromogenic labels (e.g., phosphorescent labels, dyes, and fluorophores) spectroscopic labels, enzyme linked labels, radioactive labels, and labeled binding proteins. Additional labels are described in U.S. Pat. No. 4,366,241, which is incorporated herein by reference. The resulting DNA-DNA or RNA-DNA hybridization products formed by hybridizing the labeled probe library and the non-normalized cDNA library can be detected visually or by instrument, depending on the label used. If the probes are labeled by radioactive isotope then the resulting hybridization products can be detected by exposing them to a phosphor imager or a photographic film, and developing the photographic film. The number of probe molecules that will bind to each member of the non-normalized cDNA library is proportional to the number of that species of probe molecules. Since the number of molecules for each species of probe is proportional to the number of species of each ORF represented in the mRNA sample, each member of the non-normalized cDNA library will be hybridized to an extent proportional to the number of species of each ORF represented in the mRNA sample. Consequently, species that are of high abundance in the RNA sample will be labeled to a proportionally higher level, while species that are of low abundance in the RNA sample will be labeled to a proportionally lower level. The relative intensity of each colony spot can be measured using the ArrayVision™ Genomics Software (Imaging Research Inc., St. Catherine, Ontario, Canada).

The detection methods used to determine where hybridization has taken place will typically depend upon the label selected above. Thus, for a fluorescent label a fluorescent detection method will typically be used. Pirrung, et al. (U.S. Pat. No. 5,143,854, 1992) describe the apparatus and mechanisms for scanning a substrate matrix using fluorescence detection, but a similar apparatus is adaptable for other optically detectable labels.

It is also possible to dispense with actual labeling if some means for detecting the amount of interaction between the probes and the cDNA members are available. This may take the form of an additional reagent which can indicate the intensity at the sites of interaction, or the sites that lack of interaction, e.g., a negative label. For the DNA-DNA or RNA-DNA interactions, locations of double strand interaction may be detected by the incorporation of intercalating dyes, or other reagents such as antibody or other reagents that recognize helix formation, see, e.g., Sheldon, et al. (U.S. Pat. No. 4,582,789, 1986), which is hereby incorporated herein by reference.

The hybridization intensity of each member or colony spot is measured and noted. The term “signal”, “signal intensity”, and “hybridization signal” have the same meaning as hybridization intensity. The hybridization intensity of each member or colony spot corresponds to the number of probes hybridized to each member or colony spot. The higher the number of probes hybridized to each member or colony spot: the higher the hybridization intensity. The hybridization intensity of each member or colony spot provides at least two information: (1) the rank of the member according to abundance relative to the other members of the non-normalized cDNA library, and (2) the relative abundance of the member relative to the other members of the non-normalized cDNA library. This information is then collected and processed so that the members are ordered or sorted, in ascending order or descending order, according to relative hybridization intensity of each. Preferably, the information is graphed with the hybridization intensity as the y-axis and the rank of the member as the x-axis (for example, see FIG. 3), or vice versa. Preferably, the information collection, ordering, and graphing are performed by computer.

For example: (1) According to the numbers for the relative abundance of mouse liver cytoplasmic mRNA provided in Table 1, if a non-normalized cDNA library of 490,500 members is constructed from mouse liver cells: there are 108,000 members (˜22% of the total) comprising 9 mRNA species, 210,000 members (˜43% of the total) comprising 700 mRNA species, and 172,500 members (˜35% of the total) comprising 11,500 mRNA species. (2) According to the numbers for the relative abundance of chicken oviduct polysomal mRNA provided in Table 1, if a non-normalized cDNA library of 190,500 members is constructed from mouse liver cells: 100,000 members (˜52% of the total) comprising 1 mRNA species, 28,000 members (˜15% of the total) comprising 7 mRNA species, and 62,500 members (˜33% of the total) comprising 12,500 mRNA species.

Based on the hybridization intensity, the members can be categorized into one or more classes of relative abundance. Each class comprises the members with the closest hybridization intensity ranking. The class with the less or least abundance is the low abundance class. The term “low expression” has the same meaning as “low abundance”. The classes other than the low abundance can similarly be categorized into an abundance class, and appropriately named to distinguish the ranking of the members within that class from the members in the other class(es). For example, 3,000 members are divided into three classes: the 1,000 members ranked with the highest hybridization intensities are categorized into the high abundance class, the 1,000 members ranked with the next highest hybridization intensities are categorized into the medium abundance class, and the 1,000 members ranked with the lowest hybridization intensities are categorized into the low abundance class.

A species of mRNA that has about 100.0 or less molecules per 100,000 total mRNA molecules is a mRNA of low abundance. Preferably, the mRNA of low abundance has about 50.0 or less molecules per 100,000 total mRNA molecules. More preferably, the mRNA of low abundance has about 25.0 or less molecules per 100,000 total mRNA molecules. Even more preferably, the mRNA of low abundance has about 10.0 or less molecules per 100,000 total mRNA molecules. Even further more preferably, the mRNA of low abundance has about 5.0 or less molecules per 100,000 total mRNA molecules. Even much further more preferably, the mRNA of low abundance has about 4.0, 3.0, 2.0 or 1.0 molecules per 100,000 total mRNA molecules. Based on the number of molecules determined for mouse liver cytoplasmic mRNA (see Table 1), the mRNA of low abundance is about 3.1 molecules per 100,000 total mRNA molecules. Based on the number of molecules determined for chicken oviduct polysomal mRNA (see Table 1), the mRNA of low abundance is about 2.6 molecules per 100,000 total mRNA molecules.

The percentage of members of a non-normalized cDNA library that are mRNA of low abundance is about 75%. Preferably, the percentage is about 67%. More preferably, the percentage is about 50%. Even more preferably, the percentage is about 40%. Based on the number of molecules determined for mouse liver cytoplasmic mRNA (see Table 1), the percentage of members of a non-normalized cDNA library that are mRNA of low abundance is about 35%. Based on the number of molecules determined for chicken oviduct polysomal mRNA (see Table 1), the percentage of members of a non-normalized cDNA library that are mRNA of low abundance is about 33%.

Based on the number of molecules determined for mouse liver cytoplasmic mRNA (see Table 1), if 75% of the total members that have the lowest hybridization intensity is chosen, then these chosen members would constitute 195,375 members (˜53% of the total) comprising 652 mRNA species, and 172,500 members (˜47% of the total) comprising 11,500 mRNA species. Within the chosen members, the ratio of the species with the most numerous members to the least numerous member is 20:1 (300 members:15 members), compared the same ration of the non-normalized cDNA library which is 8,333:1. This represents a more than 400 fold increase of the ratio. If 35% of the total members that have the lowest hybridization intensity is chosen, then these chosen members would constitute 147,150 members (100% of the total) comprising 9,830 mRNA species. Within the chosen members, the ratio of the species with the most numerous members to the least numerous member is 1:1 (15 members:15 members), compared the same ration of the non-normalized cDNA library which is 8,333:1. This represents a more than 8,000 fold increase of the ratio.

An aspect of the present invention comprises a method of reducing the members of a whole or part of a non-normalized cDNA library. These members of a whole or part of a non-normalized cDNA library is a group of members. The method comprises selecting a sub-group of members from the group, and identifying the members of the group that are not represented within the sub-group of members selected. Preferably, the identifying comprises: (i) constructing a labeled probe library from the sub-group of members; (ii) hybridizing the labeled probe library to the group of members; (iii) identifying each member of the group of members that is not hybridized to by the labeled probe library.

The sub-group can consist of between one member to one half of the total number of members of the group. In the interest of efficiency, the higher the hybridization intensities of the group, the fewer the number of members selected for the sub-group.

For example, from a non-normalized cDNA library of 1,000 members is selected a sub-group of 100 members. A labeled probe library is constructed from the 100 members, which is then used to probe the non-selected 900 members. Assume of the 900 members, the labeled probe library hybridizes with 700 members and does not hybridize with 200 members. This means the species represented within the 700 members are all represented with the selected sub-group of 100 members, and the species represented within the 200 members are not represented with the selected sub-group of 100 members. Consequently, by pooling the 100 members of the selected sub-group and the 200 members, the species of which are distinct from the species of the sub-group, a collection of 300 members is formed. This collection of 300 members has at least one member of each species represented within the original 1,000 members. This method reduces the redundancy within a library, and normalizes the library or brings the library closer to normalization.

In the example shown, the same method can be repeated on one or both of the selected sub-group of 100 members (e.g., further selecting 10 members to make probes to hybridize the 90 non-selected members), and the 200 members not represented within the sub-group (e.g., selecting 20 members to make probes to hybridize the 180 non-selected members). The process can be repeated one or more times. By repeating this process the number of members that represent the total number of species represented in the original group of members is repeatedly reduced until (1) a collection is achieved where each species is represented by one member, (2) the final sub-group selected consists of one member, and/or (3) the number of members in the collection is small enough so that every member can be conveniently sequenced.

Selection of the members of a sub-group can be random or purposive. Preferably, a purposive selection is made to deliberately decrease the redundancy of species within the selected members of a sub-group. Such a purposive selection can be based on the premise that members of the same species have a higher likelihood of having the same or similar hybridization intensity using the labeled probe library constructed from the RNA sample (and consequently have a higher probability of ranking closest to each other). When selecting members of a sub-group from a group, selected members as far ranked from each other in terms of hybridization intensity. Such a selection criterion increases the likelihood of decreasing the redundancy of species within the selected members of a sub-group. For example, when selecting 2 members from a group of 20 members, select the highest and lowest ranked members of the 20 members (i.e., the 1st and 20th ranked members). For example, when selecting 3 members from a group of 30 members, select the highest, middle, and lowest ranked members of the 30 members (i.e., the 1st, 30th, and 15th or 16th ranked members). For example, when selecting 4 members from a group of 40 members, select the 1 st, 14th, 27th, and 40th ranked members.

The members of the group of members of said non-normalized cDNA library represented in low amounts by said RNA sample can be pooled into a collection. The collection can be a collection of separated members or a collection where the members are mixed in a solution or suspension. The collection does not contain member(s) ruled out as a result of their discovered redundancy by the method described above. The collection can also include redundant members or clones of the same species of mRNA or cDNA. Preferably, the ratio of the number of members of the most prevalent species of mRNA or cDNA to the number of the least prevalent species of mRNA or cDNA in a collection is not more than 100:1. More preferably, the ratio of the number of members of the most prevalent species of mRNA or cDNA to the number of the least prevalent species of mRNA or cDNA in a collection is not more than 50:1. Even more preferably, the ratio of the number of members of the most prevalent species of mRNA or cDNA to the number of the least prevalent species of mRNA or cDNA in a collection is not more than 25:1. Even further more preferably, the ratio of the number of members of the most prevalent species of mRNA or cDNA to the number of the least prevalent species of mRNA or cDNA in a collection is not more than 10:1. Even much further more preferably, the ratio of the number of members of the most prevalent species of mRNA or cDNA to the number of the least prevalent species of mRNA or cDNA in a collection is not more than 5:1. Even greater much further more preferably, the ratio of the number of members of the most prevalent species of mRNA or cDNA to the number of the least prevalent species of mRNA or cDNA in a collection is not more than 2:1. Most preferably, the ratio of the number of members of the most prevalent species of mRNA or cDNA to the number of the least prevalent species of mRNA or cDNA in a collection is not more than 1:1. In addition, preferably, number of the least prevalent species of mRNA or cDNA in a collection is one.

For the purpose of this invention, a normalized library is not necessarily perfectly normalized: in which there is only exactly one member per species in the library. A preferred normalized library comprises every species, represented in the library, only represented by one member in the library. The most preferred normalized library comprises every species from an RNA sample represented in the library, wherein each species is only represented by one member in the library. The most preferred normalized cDNA library comprises every species from an RNA sample represented in the library, wherein each species is only represented by one member in the library, wherein each cDNA is a full-length clone of the structural gene or ORF of that mRNA species.

The method can further comprise: sequencing every member of said group members of said non-normalized cDNA library represented in low amounts by said RNA sample and every member of every sub-group selected prior to said pooling, wherein a sufficient number of nucleotides are sequenced to identify members that are represented by more than once; and pooling every unique member determined by said sequencing. Every member of a group or collection can be conveniently sequenced when it is faster and/or more economical to sequence every member than to reduce redundancy using the hybridization process. The sequence of a polynucleotide or insert can be determined by a standard method, for example, by dideoxy termination using double stranded templates (Sanger, et al., Proc. Natl. Acad. Sci. USA 74:5463-7, 1977). Once the sequence of an insert is obtained, the sequence of an entire ORF of a gene can be determined by probing filters containing full-length cDNAs from the cDNA library with the inserts labeled with radioactive, fluorescent, or enzyme molecules. The sequences of an entire ORF of a gene can also be determined by RT-PCR (Methods Mol. Biol. 89:333-58, 1998).

The method lends itself to automation whereby host cells containing members of the non-normalized cDNA library can be grown on support. The method also lends itself to be practiced in an array or mircoarray format.

A collection comprising a normalized cDNA library generated from one cell type or tissue of one organism using the method of the present invention can be used to generate a labeled probe library of every member of the library. The labeled probe library can be used to identify every redundant member in a non-normalized or normalized cDNA library generated from another cell type or tissue of the same organism in order to generate a normalized cDNA library from two cell types or tissues of one organism. The procedure can be expanded to generate a normalized cDNA library from one or more cell types or tissues of one organism. Using every cell type and/or tissue of an organism, a normalized cDNA library of every mRNA transcribed by the organism can be generated. By using organisms, of the same species, at different stages of development or of different genotype and/or phenotype, a normalized cDNA library of every mRNA transcribed by the species can be generated.

There are several advantages to the present invention. The ability to obtain a normalized cDNA library of mRNA of low abundance after one round of hybridization is a major cost and time saving step over existing technologies. In addition, the present invention is most efficient at obtaining and identifying the cDNA of mRNA of low abundance, which are the mRNA of most interest. Automated high-throughput of the method means that the invention can be rapidly practiced in obtaining the normalized cDNA library of many organisms in a fast and efficient fashion. Furthermore, the use of a non-normalized full-length cDNA library does not bias against the accuracy or efficiency of the method. A normalized full-length cDNA library obviates the need to identify and clone a full-length gene using an EST.

The following examples further illustrate the present invention. These examples are intended merely to be illustrative of the present invention and are not to be construed as being limiting.

### EXAMPLES

### EXAMPLE 1

### Normalization of a cDNA Library of 100,000 Members

The following is a method for normalizing cDNA clones and selecting low abundant genes in any cDNA library (see FIG. 3). It is comprises the following main steps, in which the use of complex RNA probes to hybridize the high-density colony array filter for the selection of all low abundant clones:

Library Construction and Arraying

After a High Quality cDNA Library is Constructed by any conventional method or specific method for enrichment of certain type of clones, the number of colonies representing the whole library are picked and stored in 384-well plates. All clones are arrayed in a format of 4×4 or 5×5 of 384 onto nylon filters. Typically, 100,000 or more clones are needed to cover 70-80% of a given expressed genome(s), and two filters in size of 22×22 cm are able to represent a whole library. The arrayed colony filters are then alkali lysis treated and the DNA in each colony is fixed locally on the filters.

RNA Probe Preparation and Hybridization

The same RNA sample that is used for cDNA library construction is used for the preparation of hybridization probes. Typically 100 μg of total RNA or 10 μg of poly RNA are used as templates for making the first strand cDNA labeled with ³³P This complex RNA probe should have the same representation of transcripts of expressed genes as of clones in the cDNA library arrayed on nylon filters. The filters containing the whole library are hybridized with the RNA probe, and the hybridization image and data are acquired by a phosphor imager.

Hybridization Analysis and Clone Selection

A computational program (ArrayVision™ Genomics Software) is used to analyze the hybridization intensities of each and every colony spot. The intensity data of all colony spots are sorted based on the level of intensity of each spot. For example, 100,000 clones of a human kidney cell cDNA library are arrayed. After hybridization of kidney RNA probes, about 3×10⁴ clones will show very low hybridization signals. One small portion of the clones will have very high hybridization intensity while most of the clones show various intermediate levels of hybridization intensities. The hybridization intensity reflects the abundance of the particular clones in the RNA sample. A high hybridization intensity reflects that RNA transcript is of a high abundance. While a low hybridization intensity reflects that RNA transcript is of a low abundance. Based on such an analysis, all clones can be arbitrarily classified into three abundance categories: high, medium, and low. About one third of the clones have very low hybridization intensities representing that these clones are of very low abundance. These clones in default should be “normalized” with a very low level of redundancy. These clones are the most difficult ones to be discovered by random library sequencing approach, and thus the most interesting. To determine the uniqueness of the clones in each class, a sample clones from each abundance classes can be sequenced. The sequences of the low abundant class could be all unique from each other. The uniqueness of sequences from the medium class will lower, while it will be even lower for the high abundant class.

Gene Specific Probe Preparation and Hybridization

In parallel with the above process, a few thousands of clones from the high and medium classes will be picked for sequence analysis, and, from which, to identify a set of unique clones which represent the highly abundant members of these two classes. These non-redundant clones will be used as mixed hybridization probes to subsequently hybridize the colony filters, and to subtract all the highly redundant clones from the library. This gene specific hybridization process will generate a second population of about 1×10⁴ clones in which redundancy is largely reduced.

Final Normalized cDNA Library

The combination of the hybridization selected clones by complex RNA probe plus gene specific probes and high quality fill-length cDNA libraries would yield high representation of clones of a given transcriptome with very minimal redundancy of clones. Due to the nature of all original clones were physically arrayed in 384-well plates and on filters, this process would have no bias toward certain type of clones, and would have no bad effects on the clones selected. From the highly refined population of 4×10⁴ clones of a given organism, more than about 2×10⁴ unique high quality full-length genes should be expected at the end of this process.

### EXAMPLE 2

### Normalization of a Human Kidney cDNA Library of 3,000 Members

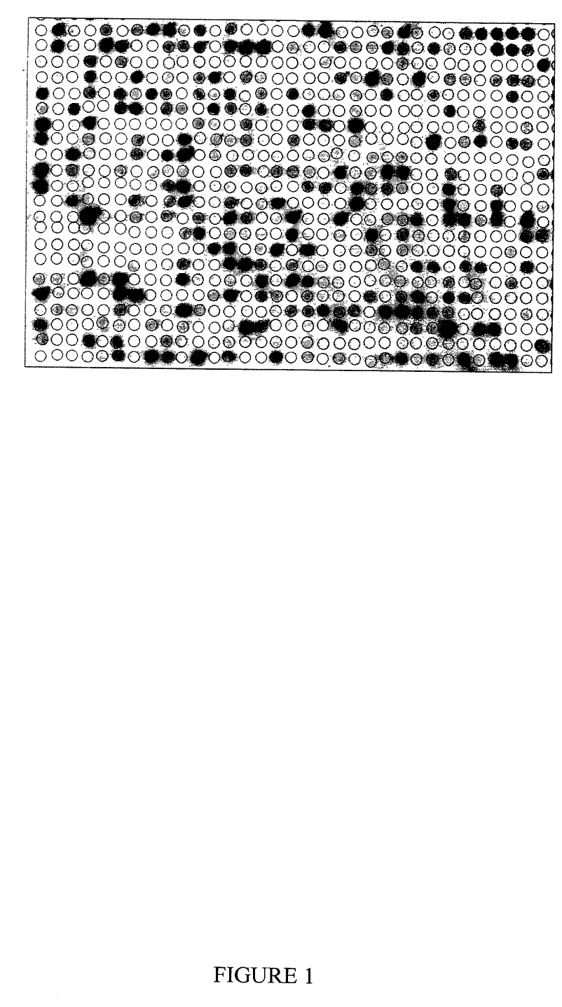

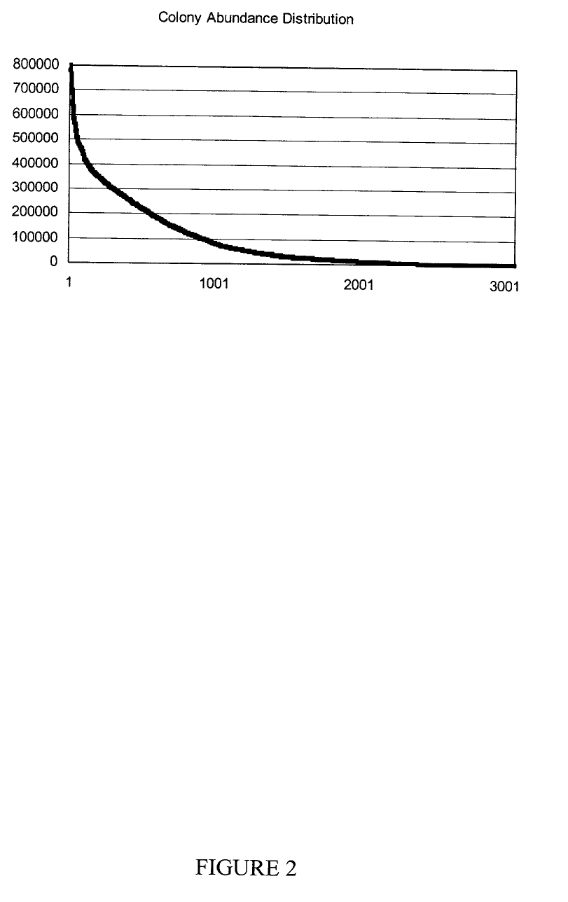

We obtained a human kidney cDNA library from Edge Biosystem (Gaithersburg, Md.). Then we picked 3,000 clones onto 384 well plates and arrayed them onto a nylon filter in size of 80×110 cm. After culturing overnight at 37° C. to amplify the clones, the colony array was processed with alkali treatment to lyse the bacterial cells and fix the DNA. We obtained a purified mRNA sample of human kidney tissue from Ambion (Austin, Tex.), and made first strand cDNA labeled with ³³P using the mRNA as the template. The complex probes were then hybridized with the high-density filter under high stringent conditions to ensure specific nucleic acid hybridization. After hybridization, the filter was exposed to a phosphor screen to capture the signals derived from the ³³P labeled probes in which specific cDNA species were hybridized to the corresponding colony spots on the filter. The screen exposed to the filter for over a week and was scanned by a phosphor imager to acquire the data of each and every colony spot. The acquired image (FIG. 1) and data were then analyzed by the ArrayVision™ Genomics Software (Imaging Research Inc., St. Catherines, Ontario, Canada). All clone spots on the filter were sorted based on hybridization intensity and plotted for abundance distribution (FIG. 2). Of the 3,000 clones analyzed, three abundance classes were arbitrarily defined as high, medium, and low. Approximately 1,000 clones were categorized in the low class, representing rare genes and indicating low redundancy within these clones.

To test the nature of transcript abundance of the clones classified into different classes, we sequenced about 200 clones from each abundant class. The sequencing results correlated with our predictions. Of the 224 clones selected from the low class, all of them represent uniquely different genes, and half of them were novel sequences without annotation matches in public databases. In contrast, the 231 clones of the medium abundant class were clustered into 144 sequence assembling contiguous representing 144 unique transcripts. The uniqueness of the medium class therefore was 80%, while the novel sequence percentage was 27%. In contrast, the uniqueness of the 200 high abundant class clones was only 7% and the novel gene percentage was 18%. The discovery rate of novel sequences increases 15 fold from the high abundant class to low abundant class. Our preliminary sequencing analysis of clones from the different classes shows that the clones from the low abundant class were indeed in default “normalized”. It is also proved that this method is a very efficient method to collect all rare genes without any obvious bias or detrimental effects to damage the integrity of the clones. From a population of 100,000 clones, at least 30,000 highly normalized clones could be generated by this approach. This refined population should contain many rare and valuable genes that were difficult to be captured otherwise. Although the test library used here was not specifically high quality in terms of full-length clones, we observed that higher percentage of full-length clones in the low class than that of the medium or high classes (Table 2). This observation implies that selecting rare clones might also increase the probability of capture more full-length cDNA clones.

TABLE 2
Statistics of DNA sequencing data.

| | | | Novel | | | | |
|---|---|---|---|---|---|---|---|
| Abundance | Total | Novel | Seq. | Annot. | Annot. | Unigene | FL Genes |
| Classes | Seq. | Clusters¹ | Rate² | Seq.³ | Cluster⁴ | Rate⁵ | (%)⁶ |
| High | 222 | 112 | 50% | 110 | 110 | 100% | 47(43) |
| Medium | 247 | 77 | 31% | 167 | 134 | 80% | 63(38) |
| Low | 246 | 7 | 3% | 199 | 13 | 7% | 54(27) |

All sequences were clustered. Each cluster was BLASTX against public protein databases.
¹Novel Clusters: Different novel sequences that do not have homology matches in the databases.
²Novel Seq. rate: The ratio of novel clusters and total sequences in each class. These ratios reflect the probabilities of finding new sequences that have not been described from each abundance class.
³Annot. Seq. (Annotated Sequences): The number of sequences that have significant but various level of homology matches in public databases.
⁴Annot. Clusters (Annotated Clusters): The number of groups of sequences that were clustered together either by sequence overlapping or by hitting onto same subject sequences in databases but in different regions.
⁵Unigene Rate: The ratio of all clusters including novel and annotated clusters over the total sequences.
⁶FL Genes (Full-Length Genes): The full-length sequences were determined by comparing only the annotated sequences to the known sequences in the databases. Most of the sequences in protein databases all close to full-length of ORF. If the sequences in the library were longer than the subject sequences at the 5′ end, we consider those sequences were potentially full-length.

In conclusion, this method allows us to select rare clones that were only 30% of the total number of clones processed but would represent 80-90% of the different transcripts expressed in a biological sample. This clone selection process will remarkably reduce the redundancy of clones to be sequenced in a large scale cDNA sequencing project with the goal of discovery of all or most expressed genes. This process also increases the probability for full-length and rare genes.

Although the invention has been described with reference to the presently preferred embodiments, it should be understood that various modifications can be made without departing from the spirit of the invention.

All publications, patents, patent applications, and web sites are herein incorporated by reference in their entirety to the same extent as if each individual patent, patent application, or web site was specifically and individually indicated to be incorporated by reference in its entirety.

What is claimed is:

1. A method for constructing a normalized cDNA library of genes of low expression, comprising:
(a) constructing a non-normalized cDNA library from an RNA sample, wherein said RNA sample contains different species of RNA of different amounts, wherein said non-normalized cDNA library contains a plurality of members;
(b) separating the members of said non-normalized cDNA library;
(c) constructing a labeled probe library from said RNA sample;
(d) hybridizing a labeled probe library to said non-normalized cDNA library, whereby there is a differential of the amount of labeled probe of said labeled probe library hybridized to each individual member of said non-normalized cDNA library;
(e) identifying the individual members of said non-normalized cDNA library hybridized with low amounts of labeled probe; and
(f) pooling the individual members of said non-normalized cDNA library identified in step (e) in a collection;
whereby said collection is said normalized cDNA library of genes of low expression.

2. The method according to claim 1, wherein said RNA sample is obtained from a cell.

3. The method according to claim 2, wherein said RNA sample is a mRNA sample.

4. The method according to claim 2, wherein said cell is an eubacteria, archaebacteria, or eukaryotic cell.

5. The method according to claim 4, wherein said eukaryotic cell is a plant cell or animal cell.

6. The method according to claim 5, wherein said plant cell is a soy, tobacco, wheat, rice, or corn cell.

7. The method according to claim 5, wherein said animal cell is a human, ape, mouse, rat, cow, pig, horse, goat, sheep, dog, cat, chicken, zebrafish, or fruitfly cell.

8. The method according to claim 7, wherein said human cell is a human kidney cell.

9. The method according to claim 1, wherein said normalized cDNA library is a normalized full-length cDNA library.

10. The method according to claim 1, wherein said constructing comprises catalyzing a reverse transcription reaction for each species of said RNA sample, wherein said catalyzing takes place under conditions permissible for catalyzing a reverse transcription reaction.

11. The method according to claim 10, wherein said catalyzing comprises:
(i) hybridizing poly-T oligonucleotide primers to said RNA sample;
(ii) adding dATP, dCTP, dGTP, dTTP, and reverse transcriptase; and
(iii) incubating said RNA sample at a temperature permissible for catalyzing a reverse transcription reaction.

12. The method according to claim 1, wherein said non-normalized cDNA library is a non-normalized full-length cDNA library.

13. The method according to claim 1, further comprising:
transforming each member of said non-normalized cDNA library into a host cell, wherein said transforming step is subsequent to said constructing and prior to said hybridizing.

14. The method according to claim 13, further comprising:
amplifying each member of said non-normalized cDNA library,
wherein said amplifying comprises growing each said host cell containing, wherein said amplifying step is subsequent to said transforming and prior to said hybridizing.

15. A method for constructing a normalized cDNA library, comprising:
(a) constructing a non-normalized cDNA library from an RNA sample, wherein said RNA sample contains different species of RNA of different amounts, wherein each member of said non-normalized cDNA library is separate from other members;
(b) identifying the relative amounts of each member of said non-normalized cDNA library represented in said RNA sample;
(c) dividing the members of said non-normalized cDNA library into groups; wherein one group of members of said non-normalized cDNA library is represented in low amounts by said RNA sample and one or more groups of members of said non-normalized cDNA library is represented in high amounts by said RNA sample;
(d) selecting one group of said one or more groups of members of said non-normalized cDNA library represented in high amounts by said RNA sample;
(e) identifying the members in said group of members that is not represented within a sub-group of members selected from said group of members;
(f) forming a group of members from the members identified in step (e) and repeating step (e) until every member of said group of members has been selected within a sub-group of members;
(g) repeating steps (d)-(f) with every group of said one or more groups of members of said non-normalized cDNA library represented in high amounts by said RNA sample;
(h) pooling the members of said group of members of said non-normalized cDNA library represented in low amounts by said RNA sample and the members of every sub-group selected in a collection;
whereby said collection is said normalized cDNA library.

16. The method according to claim 15, wherein said RNA sample is obtained from a cell.

17. The method according to claim 16, wherein said RNA sample is a mRNA sample.

18. The method according to claim 16, wherein said cell is an eubacteria, archaebacteria, or eukaryotic cell.

19. The method according to claim 18, wherein said eukaryotic cell is a plant cell or animal cell.

20. The method according to claim 19, wherein said plant cell is a soy, tobacco, wheat, rice, or corn cell.

21. The method according to claim 19, wherein said animal cell is a human, ape, mouse, rat, cow, pig, horse, goat, sheep, dog, cat, chicken, zebrafish, or fruitfly cell.

22. The method according to claim 21, wherein said human cell is a human kidney cell.

23. The method according to claim 15, wherein said normalized cDNA library is a normalized full-length cDNA library.

24. The method according to claim 15, wherein said constructing comprises catalyzing a reverse transcription reaction for each species of said RNA sample, wherein said catalyzing takes place under conditions permissible for catalyzing a reverse transcription reaction.

25. The method according to claim 24, wherein said catalyzing comprises:
(i) hybridizing poly-T oligonucleotide primers to said RNA sample;
(ii) adding dATP, dCTP, dGTP, dTTP, and reverse transcriptase; and
(iii) incubating said RNA sample at a temperature permissible for catalyzing a reverse transcription reaction.

26. The method according to claim 15, wherein said non-normalized cDNA library is a non-normalized full-length cDNA library.

27. The method according to claim 15, further comprising:
transforming each member of said non-normalized cDNA library into a host cell, wherein said transforming step is subsequent to said constructing and prior to said identifying of step (b).

28. The method according to claim 27, further comprising:
amplifying each member of said non-normalized cDNA library,
wherein said amplifying comprises growing each said host cell containing,
wherein said amplifying step is subsequent to said transforming and prior to said identifying of step (b).

29. The method according to claim 15, wherein said identifying of step (b) comprises:
(i) constructing a labeled probe library from said RNA sample;
(ii) hybridizing said labeled probe library to said non-normalized cDNA library;
(iii) identifying the relative amounts of labeled probe hybridized to each member of said non-normalized cDNA library.

30. The method according to claim 15, wherein said identifying of step (e) comprises:
(i) constructing a labeled probe library from said sub-group of members;
(ii) hybridizing said labeled probe library to said group of members;
(iii) identifying each member of said group of members that is not hybridized to by said labeled probe library.

31. The method according to claim 15, further comprising:
sequencing every member of said group members of said non-normalized cDNA library represented in low amounts by said RNA sample and every member of every sub-group selected prior to said pooling, wherein a sufficient number of nucleotides are sequenced to identify members that are represented by more than once; and
pooling every unique member determined by said sequencing.

32. A method for constructing a normalized cDNA library of genes of low expression, comprising:
(a) constructing a non-normalized cDNA library from an RNA sample, wherein said RNA sample contains different species of RNA of different amounts, wherein each member of said non-normalized cDNA library is separate from other members;
(b) identifying the relative amounts of each member of said non-normalized cDNA library represented in said RNA sample;
(c) pooling the members of said group of members of said non-normalized cDNA library represented in low amounts by said RNA sample in a collection;
whereby said collection is said normalized cDNA library of genes of low expression.

33. A normalized cDNA library generated by the method of claim 1.

34. A normalized cDNA library generated by the method of claim 8.

35. A normalized cDNA library generated by the method of claim 15.

36. A normalized cDNA library generated by the method of claim 32.
